(19) United States
(12) Patent Application Publication
(10) Pub. No.: US 2026/0038669 A1
(43) Pub. Date: Feb. 5, 2026

(54) SYSTEMS AND METHODS FOR PROVIDING PULSE AMPLITUDE MODULATION (PAM)-ENCODED SURGICAL IMAGING DATA

(71) Applicant: Stryker Corporation, Portage, MI (US)

(72) Inventors: Marc ANDRÉ, Spiegel b. Bern (CH); Thomas Darryl NEWTON, Vancouver (CA)

(73) Assignee: Stryker Corporation, Portage, MI (US)

(21) Appl. No.: 19/283,938

(22) Filed: Jul. 29, 2025

Related U.S. Application Data
(60) Provisional application No. 63/677,272, filed on Jul. 30, 2024.

(51) Int. Cl. G16H 30/20; A61B 90/00; G16H 30/40

(52) U.S. Cl. CPC G16H 30/20; A61B 90/361; G16H 30/40

(57) ABSTRACT

An exemplary system for acquiring surgical imaging data comprises a surgical imaging device for generating Pulse Amplitude Modulation (PAM)-encoded surgical imaging data, wherein the PAM-encoded surgical imaging data comprises a plurality of pulses, and each pulse of the plurality of pulses is configured to have one of more than two amplitude levels; an imaging processor for receiving data corresponding to the PAM-encoded surgical imaging data; and a galvanic isolation component between the surgical imaging device and the imaging processor.

### CROSS-REFERENCE TO RELATED APPLICATION

This application claims the benefit of U.S. Provisional Application No. 63/677,272, filed Jul. 30, 2024, the entire contents of which is incorporated herein by reference.

### FIELD

The present invention generally relates to medical imaging, and more specifically to providing and transmitting pulse amplitude modulation (PAM)-encoded surgical imaging data.

### BACKGROUND

Medical systems, instruments and tools are utilized pre-surgery, during surgery, and post-operatively for various purposes. Some of these medical tools may be used in what are generally termed as endoscopic procedures or open field procedures. For example, endoscopy allows internal features of the body of a patient to be viewed without the use of traditional, fully invasive surgery. Endoscopic imaging systems incorporate endoscopes to enable a surgeon to view a surgical site, and endoscopic tools enable minimally invasive surgery at the site. Such tools may be, for example, shaver-type devices which mechanically cut bone and hard tissue, or radio frequency (RF) probes which are used to remove tissue via ablation or to coagulate tissue to minimize bleeding at the surgical site, for example.

In endoscopic surgery, the endoscope is placed in the body at the location at which it is necessary to perform a surgical procedure. Other surgical instruments, such as the endoscopic tools mentioned above, are also placed in the body at the surgical site. A surgeon views the surgical site through the endoscope to manipulate the tools to perform the desired surgical procedure. Some endoscopes are usable along with a camera head for the purpose of capturing and processing the images received by the endoscope. An endoscopic camera system typically includes a camera head connected to a camera control unit (CCU) by a cable. The CCU processes input image data received from the image sensor of the camera via the cable and then outputs the image data for display. The resolution and frame rates of endoscopic camera systems are ever increasing, and each component of the system must be designed accordingly.

Another type of medical imager that can include a camera head connected to a CCU by a cable is an open-field imager. Open-field imagers can be used to image open surgical fields, for example, for visualizing blood flow in vessels and related tissue perfusion during plastic, microsurgical, reconstructive, and gastrointestinal procedures.

Existing imaging systems (e.g., endoscopic imaging systems, open-field imaging systems, and other types of medical imaging systems) may transmit medical imaging data using binary signals (also referred to as Non-Return-to-Zero (NRZ) signals). An NRZ signal uses low and high signal levels to represent the 1/0 information of a digital logic signal and thus can only transmit 1 bit (i.e., a 0 or a 1) of information per signal symbol period. In order to support the high data rate (which necessitates higher-frequency signals) and maintain the signal integrity, a high-quality cable is required. Further, the data signal may be difficult to galvanically isolate because isolating high-speed data while maintaining power transmission and signal integrity is challenging.

### SUMMARY

Described herein are devices, systems, and methods for providing and transmitting Pulse Amplitude Modulation (PAM)-encoded surgical imaging data. A PAM-encoded signal comprises a plurality of pulses and each pulse of the plurality of pulses is configured to have one of more than two amplitude levels (e.g., four amplitude levels, eight amplitude levels, sixteen amplitude levels). Comparing to a conventional binary signal, a PAM signal can transmit information at a lower signal frequency for the same amount of data or transmit information at a higher data rate over the same amount of bandwidth. PAM encoding has not been used in the surgical context (e.g., for transmitting surgical imaging data). While PAM is a known technique in digital communication, its application to surgical imaging systems is non-obvious due to domain-specific requirements. For example, one of the data communication standards supporting PAM encoding is the A-PHY standard, but the A-PHY standard is tailored for automotive systems. Adopting PAM encoding for transmission of surgical imaging data, such as A-PHY or any other standards supporting PAM encoding, can be advantageous because PAM data transmission can allow for better image quality, a more reliable and cheaper camera cable at reduced cost, and more effective and cheaper galvanic isolation, which are important for medical settings.

An exemplary system can comprise a surgical imaging device for generating PAM-encoded surgical imaging data and an imaging processor for receiving data corresponding to the PAM-encoded surgical imaging data. The PAM-encoded surgical imaging data comprises a plurality of pulses and each pulse of the plurality of pulses is configured to have one of more than two amplitude levels (e.g., four amplitude levels, eight amplitude levels, sixteen amplitude levels). A conventional binary signal (also referred to as a NRZ signal) uses low and high signal levels to represent the 1/0 information of a digital logic signal. NRZ can only transmit 1 bit (i.e., a 0 or a 1) of information per signal symbol period. In contrast, a PAM-encoded signal comprises a plurality of pulses and each pulse of the plurality of pulses is configured to have one of more than two amplitude levels (e.g., four amplitude levels, eight amplitude levels, sixteen amplitude levels, or the like). For example, a PAM4 signal has four voltage levels and thus four amplitude levels. Thus, each signal level can represent 2 bits of logic information per signal symbol period. Accordingly, a PAM4 signal can transmit information at a lower signal frequency for the same amount of data or transmit information at a higher data rate over the same amount of bandwidth.

In some examples, the PAM-encoded surgical imaging data may be encoded and transmitted in accordance with the A-PHY standard. The system may comprise an encoder and a decoder on the two sides of the camera cable connection.

The exemplary system can further comprise a galvanic isolation component. In some examples, the galvanic isolation component may be located between the surgical imaging device and the imaging processor. Galvanic isolation can be achieved more easily with PAM-encoded signals because the signal frequency can be lower. As described herein, the topology, location, and mechanism of the galvanic isolation component may vary, as long as it is capable of properly transmitting the PAM-encoded signals at the required frequency and providing the needed isolation voltage. There are multiple locations in the data transmission and processing path that the isolation can take place.

Examples of the present disclosure provide several technical advantages. PAM data transmission can allow higher camera data rates and/or lower signal frequencies. PAM data transmission can also allow a more reliable camera cable at reduced cost, as well as reducing the cost of associated galvanic isolation. For example, while the data rate under CoaXPress is currently capped at 12 Gb/s (for a single wire running at 6 GHz), the current A-PHY standard can support as much as 14.4 Gb/s (for a single wire running at 2 GHz with PAM16) and may support data rates up to 32 Gb/s, thus supporting higher frame rate and/or resolution. Further, the A-PHY standard can provide a lower packet error rate and be fully implemented in hardware without requiring dedicated software stack. It can provide high-speed downlink and aggregation to support multiple 4K cameras and displays.

Further, the frequency on the transmission line can be lower, which makes it easier to build the cable (e.g., due to fewer signal integrity issues, fewer cable quality issues, and enhanced noise immunity) and longer cable lengths can be achieved. The system can be cheaper by cutting down the cost associated with optical transceivers.

Further, galvanic isolation (as needed for medical devices) is simpler due to the lower signal frequency. For example, it can be very difficult and expensive to isolate a conventional 12 Gb/s CoaXPress signal, but isolation can be achieved for a 14.4 Gb/s A-PHY signal with a low-cost magnetic transformer. This allows the use of a simpler cable (e.g., coaxial conductors or twisted pair) and galvanic isolation using standard magnetic transformers.

An exemplary system for acquiring surgical imaging data comprises: a surgical imaging device for generating Pulse Amplitude Modulation (PAM)-encoded surgical imaging data, wherein: the PAM-encoded surgical imaging data comprises a plurality of pulses, and each pulse of the plurality of pulses is configured to have one of more than two amplitude levels; an imaging processor for receiving data corresponding to the PAM-encoded surgical imaging data; and a galvanic isolation component between the surgical imaging device and the imaging processor.

In some examples, each pulse of the plurality of pulses is configured to have one of four amplitude levels, one of eight amplitude levels, or one of sixteen amplitude levels. In some examples, the PAM-encoded surgical imaging data is encoded and transmitted in accordance with the A-PHY standard.

The surgical imaging device may comprise: one or more imaging sensors for generating original surgical imaging data; and an encoder for encoding the original surgical imaging data to obtain the PAM-encoded surgical imaging data. The encoder may be a programmable device or an application-specific integrated circuit (ASIC).

In some examples, the surgical imaging device comprises one or more imaging sensors for generating the PAM-encoded surgical imaging data.

In some examples, the system further comprises a cable for transmitting the PAM-encoded surgical imaging data from the surgical imaging device, wherein the cable comprises a transmission line. The transmission line can be a twisted pair transmission line or a coaxial transmission line. The transmission line can be configured to transmit power from the imaging processor to the surgical imaging device. The cable can be configured to transmit a control signal from the imaging processor to the surgical imaging device. The control signal and the PAM-encoded signal can be transmitted as a plurality of data packets along the transmission line. The plurality of packets may have the same structure. At least one of the plurality of packets can be configured to be retransmitted using a retransmission buffer.

In some examples, the system further comprises a light source, wherein the surgical imaging device is configured to output one or more light pulses, and wherein synchronization of the light source and the one or more light pulses is controlled by a synchronization signal on the transmission line.

In some examples, the galvanic isolation component comprises a magnetic transformer or one or more CMOS isolation circuits. In some examples, the system further comprises a decoder for decoding the PAM-encoded surgical imaging data. The galvanic isolation component can be located between the surgical imaging device and the decoder or between the decoder and the imaging processor. In some examples, the surgical imaging data comprises at least one of pixel data and voxel data.

It will be appreciated that any one or more of the above aspects, features, examples, and options can be combined. It will be appreciated that any one of the examples and/or options described in view of system apply equally to the imaging device, imaging controller or method, and vice versa.

### BRIEF DESCRIPTION OF THE FIGURES

The invention will now be described, by way of example only, with reference to the accompanying drawings, in which:

FIG. 1A shows an example of an endoscopic camera system;

FIG. 1B shows an example of an open-field camera system;

FIG. 2 illustrates an exemplary system for acquiring surgical imaging data;

FIG. 3A illustrates a comparison of a binary signal and a PAM-encoded signal;

FIG. 3B illustrates transmission of information using an NRZ signal v. a PAM4 signal;

FIG. 4A illustrates an exemplary encoder;

FIG. 4B illustrates an exemplary decoder;

FIGS. 5A-G illustrate exemplary galvanic isolation schemes;

FIG. 6 is an illustrative depiction of an exemplary fluorescence imaging system;

FIG. 7 is an illustrative depiction of an exemplary illumination module of a fluorescence imaging system; and

FIG. 8 is an exemplary camera module of a fluorescence imaging system.

### DETAILED DESCRIPTION

Reference will now be made in detail to implementations and examples of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Described herein are devices, systems, and methods for providing and transmitting Pulse Amplitude Modulation (PAM)-encoded surgical imaging data. A PAM-encoded signal comprises a plurality of pulses and each pulse of the plurality of pulses is configured to have one of more than two amplitude levels (e.g., four amplitude levels, eight amplitude levels, sixteen amplitude levels). Comparing to a conventional binary signal, a PAM signal can transmit information at a lower signal frequency for the same amount of data or transmit information at a higher data rate over the same amount of bandwidth. PAM encoding has not been used in the surgical context (e.g., for transmitting surgical imaging data). For example, one of the data communication standards supporting PAM encoding is the A-PHY standard, but the A-PHY standard is tailored for automotive systems. Adopting PAM encoding for transmission of surgical imaging data can be advantageous because PAM data transmission can allow for better image quality, a more reliable and cheaper camera cable at reduced cost, and more effective and cheaper galvanic isolation, which are important for medical settings.

An exemplary system can comprise a surgical imaging device for generating PAM-encoded surgical imaging data and an imaging processor for receiving data corresponding to the PAM-encoded surgical imaging data. The PAM-encoded surgical imaging data comprises a plurality of pulses and each pulse of the plurality of pulses is configured to have one of more than two amplitude levels (e.g., four amplitude levels, eight amplitude levels, sixteen amplitude levels). A conventional binary signal (also referred to as a Non-Return-to-Zero (NRZ) signal) uses low and high signal levels to represent the 1/0 information of a digital logic signal. NRZ can only transmit 1 bit (i.e., a 0 or a 1) of information per signal symbol period. In contrast, a PAM-encoded signal comprises a plurality of pulses and each pulse of the plurality of pulses is configured to have one of more than two amplitude levels (e.g., four amplitude levels, eight amplitude levels, sixteen amplitude levels, or the like). For example, a PAM4 signal has four voltage levels and thus four amplitude levels. Thus, each signal level can represent 2 bits of logic information per signal symbol period. Accordingly, a PAM4 signal can transmit information at a lower signal frequency for the same amount of data or transmit information at a higher data rate over the same amount of bandwidth.

In some examples, the PAM-encoded surgical imaging data may be encoded and transmitted in accordance with the A-PHY standard. The system may comprise an encoder and a decoder on the two sides of the camera cable connection.

The exemplary system can further comprise a galvanic isolation component. In some examples, the galvanic isolation component may be located between the surgical imaging device and the imaging processor. Galvanic isolation can be achieved more easily with PAM-encoded signals because the signal frequency can be lower. As described herein, the topology, location, and mechanism of the galvanic isolation component may vary, as long as it is capable of properly transmitting the PAM-encoded signals at the required frequency and provide the needed isolation voltage. There are multiple locations in the data transmission and processing path that the isolation can take place.

Examples of the present disclosure provide several technical advantages. PAM data transmission can allow higher camera data rates and/or lower signal frequencies. PAM data transmission can also allow a more reliable camera cable at reduced cost, as well as reducing the cost of associated galvanic isolation. For example, while the data rate under CoaXPress is currently capped at 12 Gb/s (for a single wire running at 6 GHZ), the current A-PHY standard can support as much as 14.4 Gb/s (for a single wire running at 2 GHz with PAM16) and may support data rates up to 32 Gb/s, thus supporting higher frame rate and/or resolution. Further, the A-PHY standard can provide a lower packet error rate and be fully implemented in hardware without requiring a dedicated software stack. It can provide high-speed downlink and aggregation to support multiple 4K cameras and displays.

Further, the frequency on the transmission line can be lower, which makes it easier to build the cable (e.g., due to fewer signal integrity issues, fewer cable quality issues, and enhanced noise immunity) and longer cable lengths can be achieved. The system can be cheaper by cutting down the cost associated with optical transceivers.

Further, galvanic isolation (as needed for medical devices) is simpler due to the lower signal frequency. For example, it can be very difficult and expensive to isolate a conventional 12 Gb/s CoaXPress signal, but isolation can be achieved for a 14.4 Gb/s A-PHY signal with a low-cost magnetic transformer. This allows the use of a simpler cable (e.g., coaxial conductors or twisted pair) and galvanic isolation using standard magnetic transformers.

In the following description of the various examples, reference is made to the accompanying drawings, in which are shown, by way of illustration, specific examples that can be practiced. It is to be understood that other embodiments and examples can be practiced, and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some examples also relates to a device for performing the operations herein. This device may be specially constructed for the required purposes, or it may comprise a general purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each connected to a computer system bus. Furthermore, the computers referred to in the specification may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

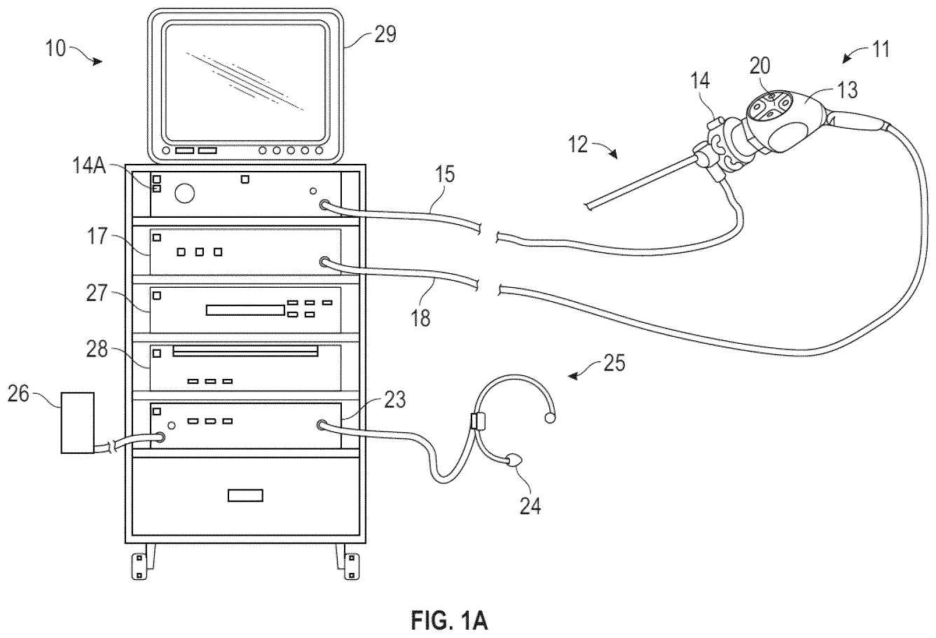

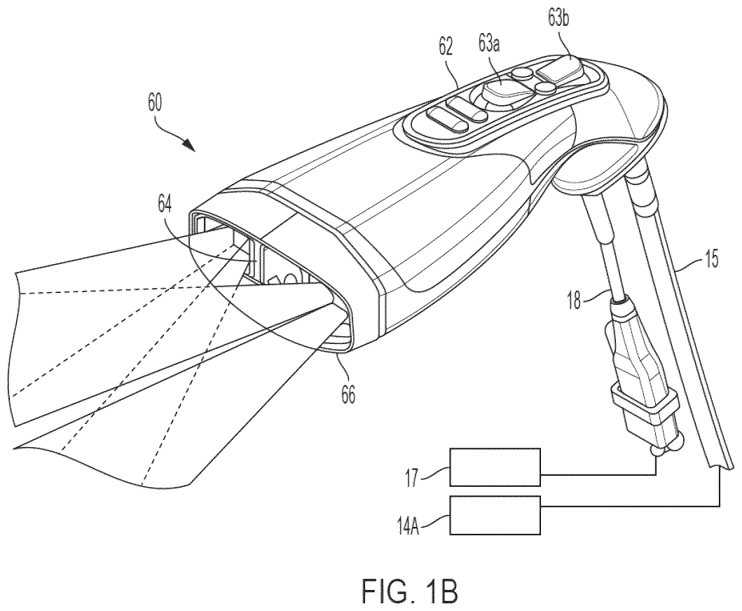

FIG. 1A shows an exemplary medical imaging system 10 that can utilize an, e.g. authenticable, data cable for connecting a medical imaging device to a medical imaging controller, according to the principles described herein. As used herein, medical imaging includes, but is not limited to, pre-operative, intra-operative, post-operative, and diagnostic imaging sessions and procedures. System 10 includes a scope assembly 11 which may be utilized in endoscopic procedures. The scope assembly 11 incorporates an endoscope or scope 12 which is coupled to an endoscopic camera head 13 by a coupler 14 located at the distal end of the camera head 13. Light is provided to the scope by a light source 14A via a light guide 15, such as a fiber optic cable. The camera head 13 is connected to a camera control unit (CCU) 17 by an electrical cable 18. Operation of the camera 13 is controlled, in part, by the CCU 17. The cable 18 conveys or transmits still and/or video image data from the camera head 13 to the CCU 17 and conveys various control signals bi-directionally between the camera head 13 and the CCU 17. In one example, the image data output by the camera head 13 is digital. The cable 18 may include a memory device for storing authentication data for authenticating the cable 18, as discussed further below. Any imaging methods as described herein may be performed after the endoscope has been pre-inserted into a body cavity.

A control or switch arrangement 20 may be provided on the camera head 13 and allows a user to manually control various functions of the system 10. These and other functions may also be controlled by voice commands using a voice-control unit 23, which is connected to the CCU 17. Optionally, voice commands are input into a microphone 24 mounted on a headset 25 worn by the surgeon and by means of a wire, or wirelessly, coupled to the voice-control unit 23. A hand-held control device 26, such as a tablet with a touch screen user interface or a PDA, may be connected to the voice control unit 23 as a further control interface. In the illustrated example, a recorder 27 and a printer 28 are also connected to the CCU 17. Additional devices, such as an image capture and archiving device, may be included in the system 10 and connected to the CCU 17. Video image data acquired by the camera head 13 and processed by the CCU 17 is converted to images, which can be displayed on a monitor 29, recorded by recorder 27, and/or used to generate static images, hard copies of which can be produced by printer 28.

FIG. 1B illustrates an open-field imaging device 60, which is another example of a type of imaging device that can be connected to an imaging controller via an, e.g. authenticable, cable, as discussed herein. Open-field imaging device 60 can be used as part of an imaging system, such as system 10 of FIG. 1A, for various purposes, including for visualizing blood flow in vessels and related tissue perfusion during plastic, microsurgical, reconstructive, and gastrointestinal procedures. As may be seen in FIG. 1B, the open-field imaging device 60 includes a control surface 62, a window frame 64 and a nosepiece 66. The open-field imaging device 60 is in this example connectable to the light source 14A via a light guide cable 15, through which the light is provided to the imaging field via ports in the window frame 64. The open-field imaging device 60 is connectable to the CCU 17 via an, e.g. authenticable, data cable 18, according to the principles described herein, which can transmit power, imaging data, and any other types of data.

The control surface 62 here includes focus buttons 63a (decreasing the working distance) and 63b (increasing the working distance) that control, e.g., outlet angles of the light beams for controlling a working distance at which the light beams substantially overlap for illuminating a target area. Other buttons on the control surface 62 may be programmable and may be used for various other functions, e.g., excitation laser power on/off, display mode selection, white light imaging white balance, saving a screenshot, and so forth. In some examples, the control surface functions can be communicated to the CCU 17 via non-imaging data communication lines in the cable 18, as discussed further below.

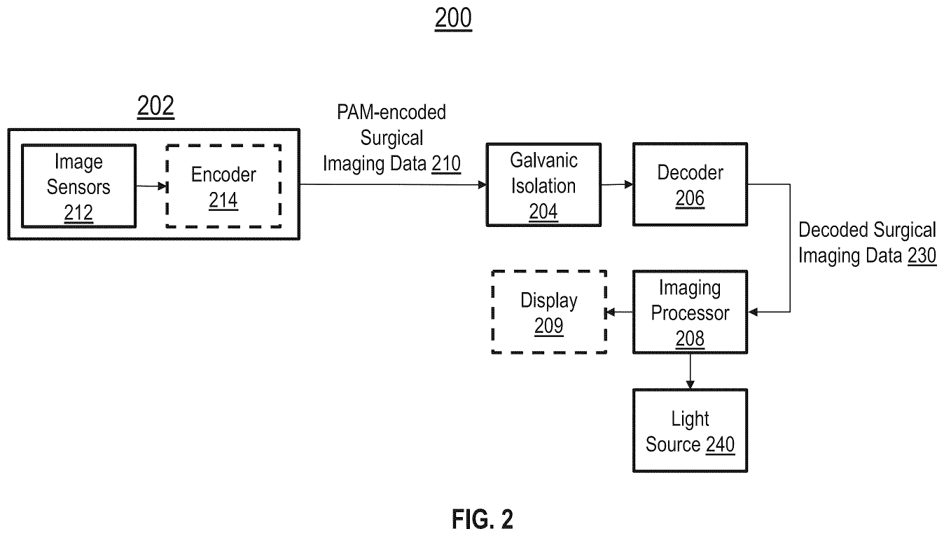

FIG. 2 illustrates an exemplary system 200 for acquiring surgical imaging data, in accordance with some examples. With reference to FIG. 2, the system 200 comprises a surgical imaging device 202, a galvanic isolation component 204, a decoder 206, and an imaging processor 208. In some examples, the system 200 further comprises a display.

The surgical imaging device 202 can generate Pulse Amplitude Modulation (PAM)-encoded surgical imaging data 210. In some examples, the surgical imaging device 202 comprises a camera head. The surgical imaging device 202 includes one or more imaging sensors 212 that are used to capture various types of visual information before, during, and after surgical procedures to assist surgeons in planning, navigating, and performing surgeries. Exemplary surgical imaging data can include endoscopic imaging data (e.g., for visualizing the inside of organs and body cavities), fluorescence imaging data (e.g., for visualizing blood flow and tissue perfusion), X-ray imaging data, computed tomography (CT) imaging data, magnetic resonance imaging (MRI) data, ultrasound imaging data, optical coherence tomography (OCT) imaging data, or any combination thereof. In some examples, the surgical imaging data comprises at least one of pixel data and voxel data.

In the depicted example, the surgical imaging device 202 includes both the imaging sensors 212 and an encoder 214. The imaging sensors 212 can generate original surgical imaging data (e.g., pixel data). The encoder 214 can receive the original surgical imaging data from the imaging sensors 212 and encode the original surgical imaging data to obtain the PAM-encoded surgical imaging data 210. The encoder 214 can be a programmable device (e.g., a Field Programmable Gate Array (FPGA) device) or an application-specific integrated circuit (ASIC). In some examples (not depicted), the surgical imaging device 202 may not include the encoder 214. Instead, the image sensors 212 can output the PAM-encoded surgical imaging data 210 directly.

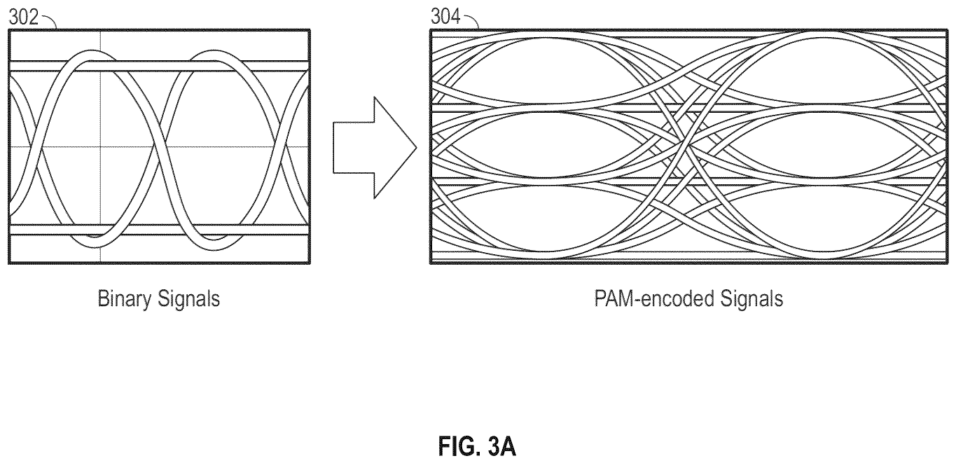

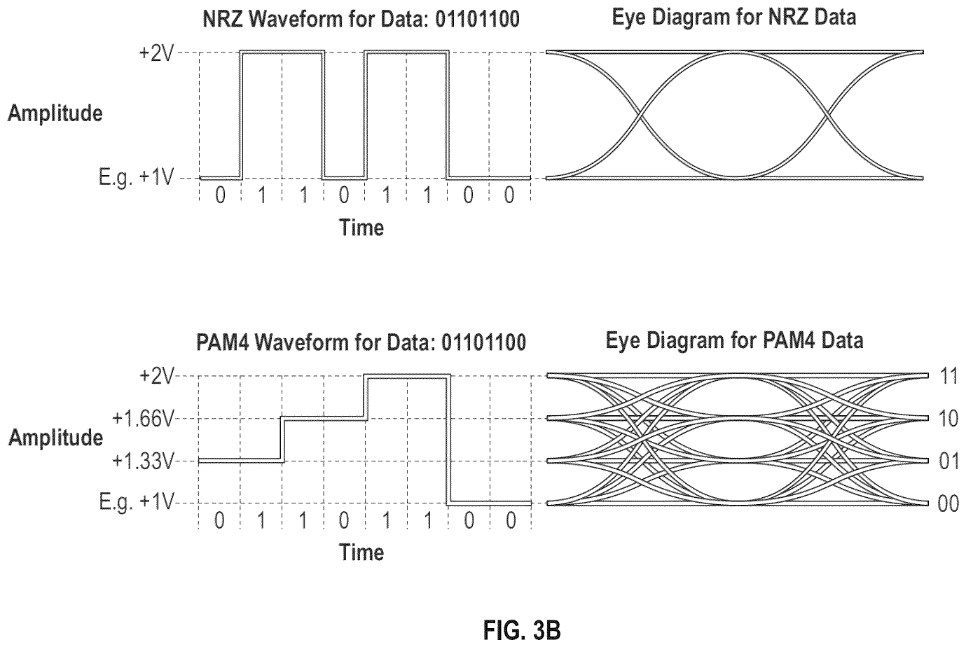

The PAM-encoded imaging data 210 comprises a plurality of pulses. Each pulse of the plurality of pulses is configured to have one of more than two amplitude levels. In some examples, the PAM-encoded surgical imaging data is encoded and transmitted in accordance with the A-PHY standard. Additional details of the PAM-encoded imaging data 210 are provided herein with reference to, for example, FIGS. 3A-B.

With reference to FIG. 2, the PAM-encoded surgical imaging data 210 can be transmitted to the decoder 206. The decoder 206 can decode the PAM-encoded surgical imaging data 210 and provide the decoded surgical imaging data 230 to the imaging processor 208. In some examples, the imaging processor 208 is a part of the CCU. The imaging processor 208 can comprise a plurality of algorithms for processing the decoded surgical imaging data 230 to enhance, analyze, and/or interpret the decoded surgical imaging data 230 to assist in surgical planning, navigation, and execution. Exemplary algorithms can include any image processing algorithms, such as image segmentation algorithms, image registration algorithms, image enhancement algorithms, image reconstruction algorithms, image fusion algorithms, image analysis and quantification algorithms, machine learning algorithms (e.g., detection algorithms, diagnosis algorithms), demosaic algorithms, or any combination thereof. The imaging processor 208 can provide results of the imaging processing to the display 209 for display.

The system can comprise a cable (not depicted) for transmitting the PAM-encoded surgical imaging data 210 (e.g., from the camera head) to the imaging processor 208 (e.g., CCU). In the depicted example, the cable may be located between the encoder 214 and the galvanic isolation component 204. The cable can comprise a transmission line. The transmission line can be, or comprise, a twisted pair transmission line (e.g., shielded or un-shielded twisted pair transmission line) or another type of transmission line that can transmit differential signals. The transmission line can be, or comprise, a coaxial transmission line, such as uCoax (micro-coax) lines. The transmission line can be configured to transmit power (e.g., as DC offset) from the imaging processor 208 to the surgical imaging device 202 in addition to transmitting the PAM-encoded surgical imaging data 210 to the imaging processor 208. Alternatively, separate transmission lines can be used for transmitting the PAM-encoded surgical imaging data 210 to the imaging processor 208 and for transmitting power to the surgical imaging device 202, respectively.

The same transmission line for transmitting the PAM-encoded surgical imaging data 210 to the imaging processor 208 can be used for transmitting one or more control signals from the imaging processor 208 to the surgical imaging device 202. Alternatively, separate transmission lines can be used for transmitting the PAM-encoded surgical imaging data 210 from the surgical imaging device 202 to the imaging processor 208 and for transmitting one or more control signals from the imaging processor 208 to the surgical imaging device 202, respectively. Control signals may be transmitted from the surgical imaging device 202 to the imaging processor 208 (e.g., from buttons, joysticks, or the like). The one or more control signals can be used to configure the surgical imaging device 202, interact with any user interface (e.g., buttons) on the surgical imaging device 202, and read additional sensors. With protocol adaption layers (PAL), protocols such as I2C, SPI or Ethernet can be used. In some examples, the one or more control signals can be used to send synchronization messages for synchronizing image acquisition with a light source 240 of the system 200, as described in detail below.

The one or more control signals and the PAM-encoded surgical imaging data 210 can be transmitted as a plurality of data packets along the same transmission line of the cable or different transmission lines. In some examples, the plurality of data packets (e.g., for transmitting PAM-encoded surgical imaging data, for transmitting control signals, or both) may have the same packet structure at a given transmission layer. For example, each of the plurality of data packets can be generated as an A-Packet, which is structured to carry the native protocol data and all information that the A-PHY Data Link Layer requires to perform its functions efficiently. The A-packet structure may be optimized for supporting multiple protocol aggregation with minimal overhead and latency. The A-Packet header may contain all required information (e.g., QoS, Priority, Destination, Protocol Type). In some examples, the A-packet structure comprises an 8-byte header including a message counter (MC) and a header Cyclic Redundancy Check (CRC), the payload, and a 4-byte tail.

In some examples, at least one of the plurality of packets can be configured to be retransmitted using a retransmission buffer. The retransmission buffer can be used to resend any corrupted packets to improve signal quality in noisy environments. The retransmission may be handled at the A-PHY level and is transparent to the upper layer. In some examples, the retransmission buffer can provide for multiple (e.g., up to three) retries.

The light source 240 can be synchronized with the image acquisition by the surgical imaging device 202. For example, a synchronization signal is used to synchronize light pulses from the light source 204 and imaging of the light pulses by the surgical imaging device 202. The synchronization signal can function as a clock. The imaging processor 208 can generate a sensor synchronization signal (e.g., a vertical sync signal), which is sent to both the camera of the surgical imaging device 202 and to the light source 240.

In some examples, the surgical imaging device 202 contains a clock frequency adjuster such as a phase-locked loop (PLL) to adjust the synchronization signal to match the clock required by the image sensors. Depending on the required accuracy of the sensor synchronization signal, this synchronization signal can be used directly by the image sensors 212.

The sensor synchronization signal can be generated on the surgical imaging device 202 and initially triggered (and monitored) with the synchronization signal from the imaging processor 208. The imaging processor 208 and the sensor synchronization generator in the surgical imaging device 202 can run on the same clock (e.g., protocol synchronization signal) and the interval of the sensor synchronization signal is a configurable clock count. In some examples, the synchronization signal is generated on the surgical imaging device 202 and transmitted to the imaging processor 208, which forwards it to the light source 240. In some examples, the synchronization signal is transmitted with a separate transmission line.

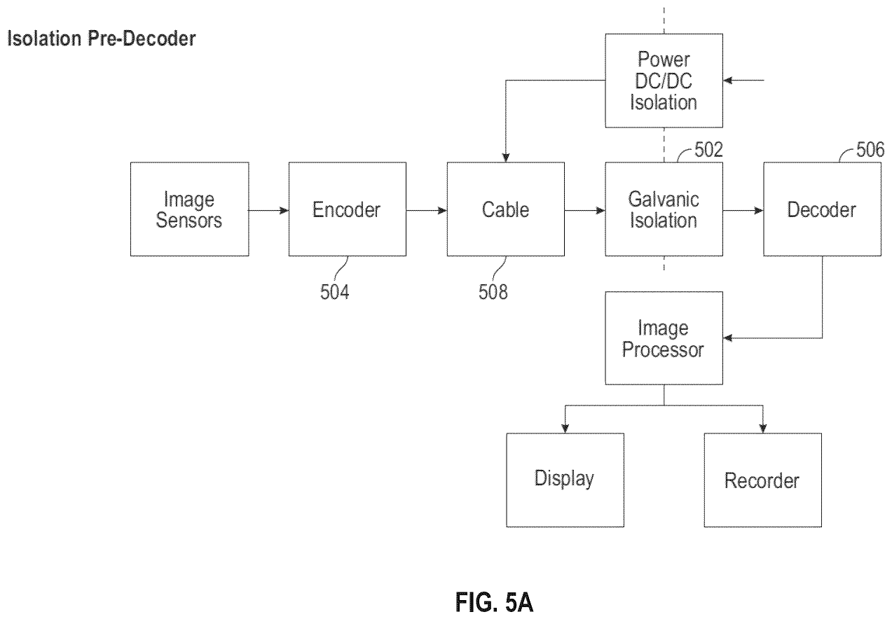

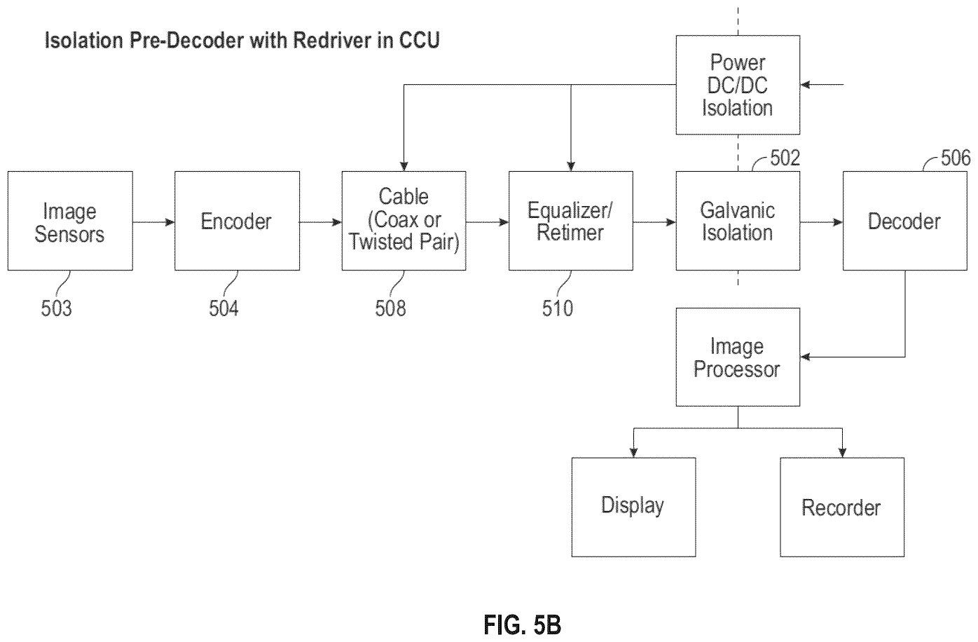

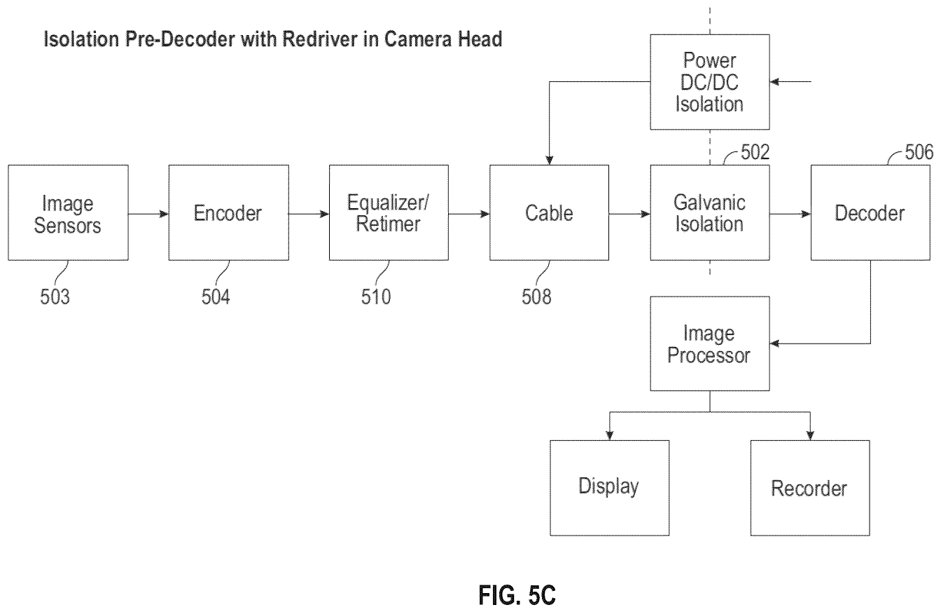

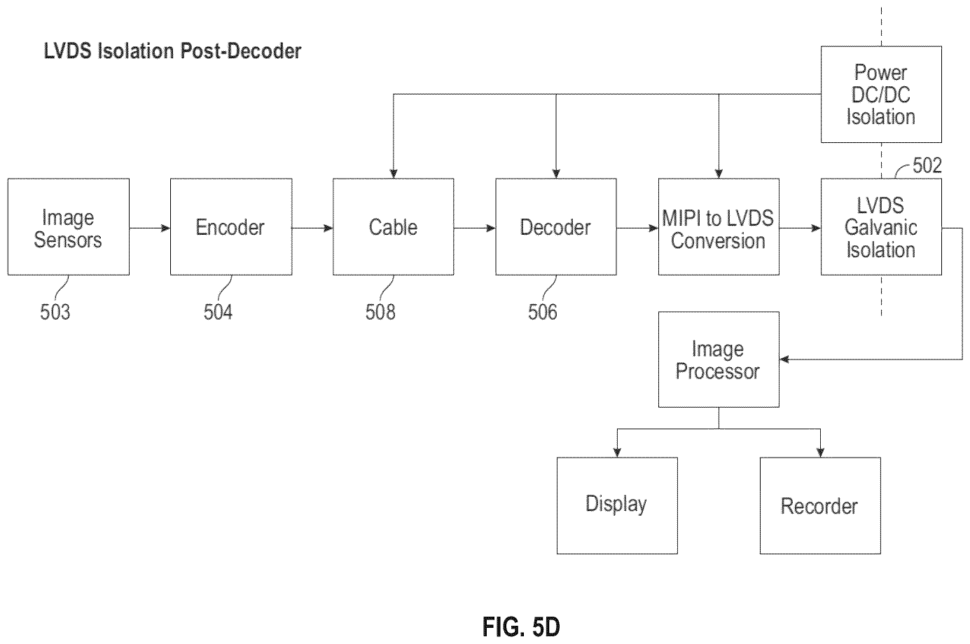

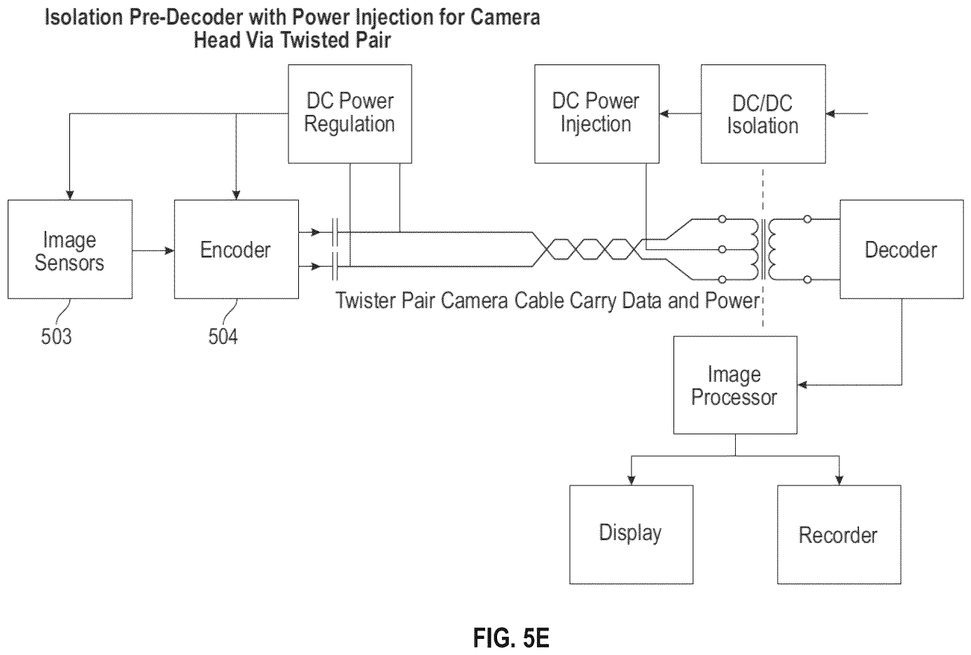

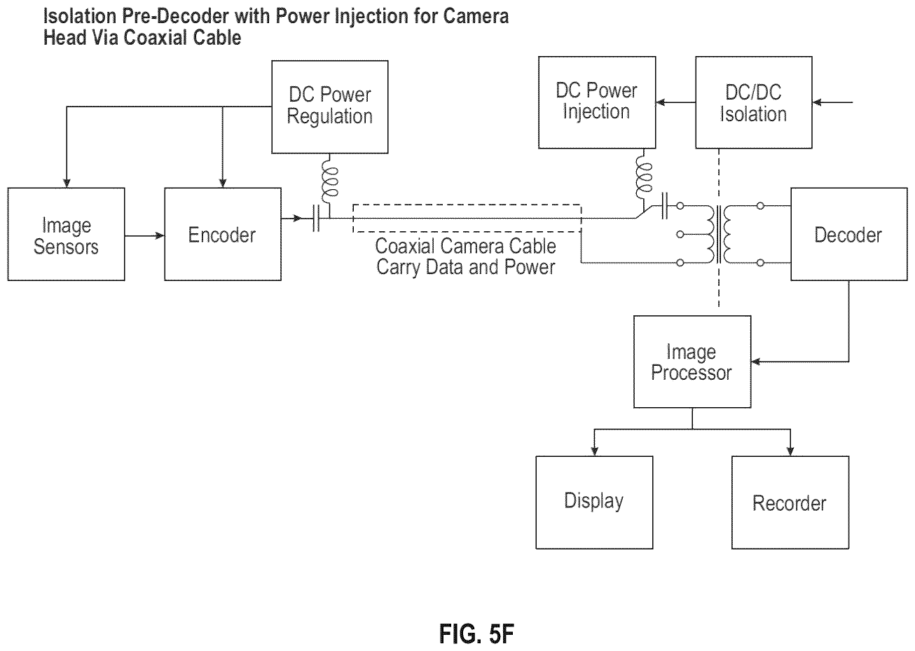

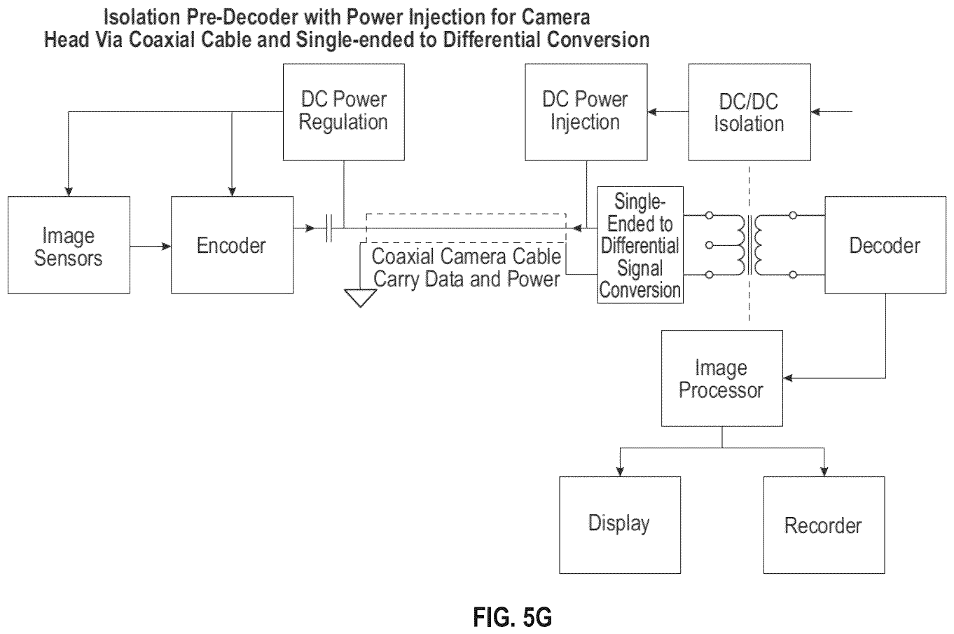

With reference to FIG. 2, the system 200 further comprises a galvanic isolation component 204 for providing galvanic isolation of the PAM-encoded surgical imaging device 202. Galvanic isolation is used to separate electrical circuits to prevent current flow between them. The galvanic isolation component 204 can ensure patient safety by isolating the surgical imaging device 202 from direct contact with other electrical components and from mains power, and to prevent signal interference. In some examples, the galvanic isolation component 204 can comprise a magnetic transformer. In some examples, the PAM-encoded surgical imaging data 210 may be deserialized to be galvanically isolated by CMOS highspeed isolation integrated circuits (e.g., LVDS isolation). In some examples, the PAM-encoded surgical imaging data 210 may be galvanically isolated using optical methods, millimeter wave transmitters, or any other suitable galvanic isolation techniques. Examples of the galvanic isolation schemes are provided herein with reference to FIGS. 5A-G.

In some examples, the galvanic isolation component 204 is located between the surgical imaging device 202 and the imaging processor 208. In the depicted example, the galvanic isolation component 204 is located between the surgical imaging device 202 and the decoder 206, and more specifically between the encoder 214 and the decoder 206. Alternatively (not depicted), the galvanic isolation component 204 may be located between the decoder 206 and the imaging processor 208. Additionally or alternatively, the imaging processor 208 may comprise multiple processors and a galvanic isolation component may be located between the multiple processors.

FIG. 3A illustrates a comparison of eye diagrams for a binary signal 302 and a PAM-encoded signal 304, in accordance with some examples. In the depicted example, the binary signal 302 has two voltage levels and thus two amplitude levels. The binary signal 302 (also referred to as an NRZ signal) thus uses low and high signal levels to represent the 1/0 information of a digital logic signal. NRZ can only transmit 1 bit (i.e., a 0 or a 1) of information per signal symbol period.

In contrast, a PAM-encoded signal (e.g., the PAM-encoded surgical imaging data 210 in FIG. 2) comprises a plurality of pulses and each pulse of the plurality of pulses is configured to have one of more than two amplitude levels (e.g., four amplitude levels, eight amplitude levels, sixteen amplitude levels, or the like). In the depicted example in FIG. 3A, the PAM-encoded signal 304 has four voltage levels and thus four amplitude levels (also referred to as PAM4). Thus, each signal level can represent 2 bits of logic information per signal symbol period and thus can transmit information as a faster rate and/or at a lower signal frequency.

FIG. 3B illustrates transmission of information using an NRZ signal v. a PAM4 signal, in accordance with some examples. As shown, a PAM4 signal can represent 2 bits of logic information per pulse and thus can transmit the same amount of data at a lower signal frequency.

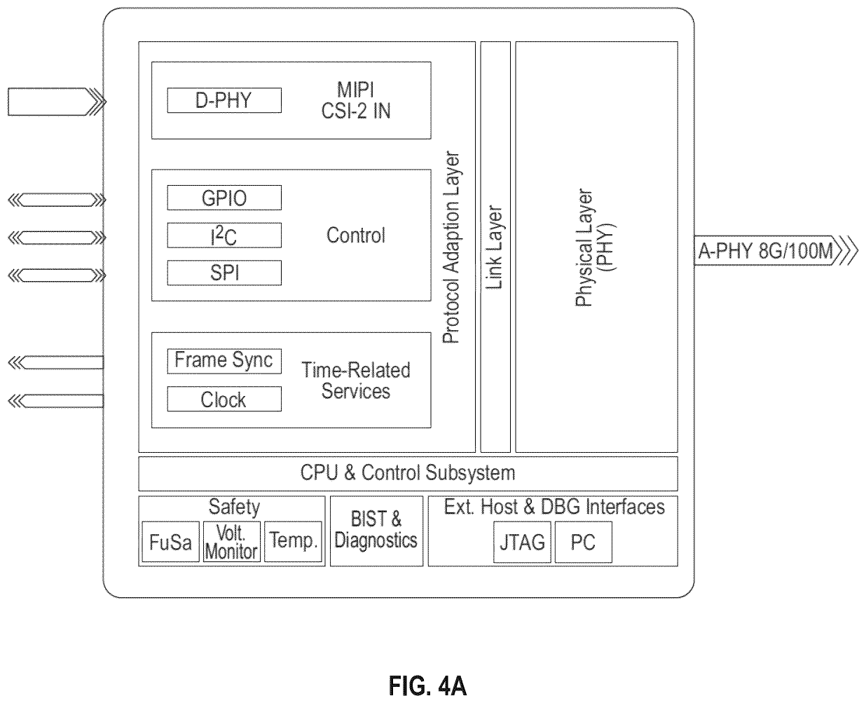

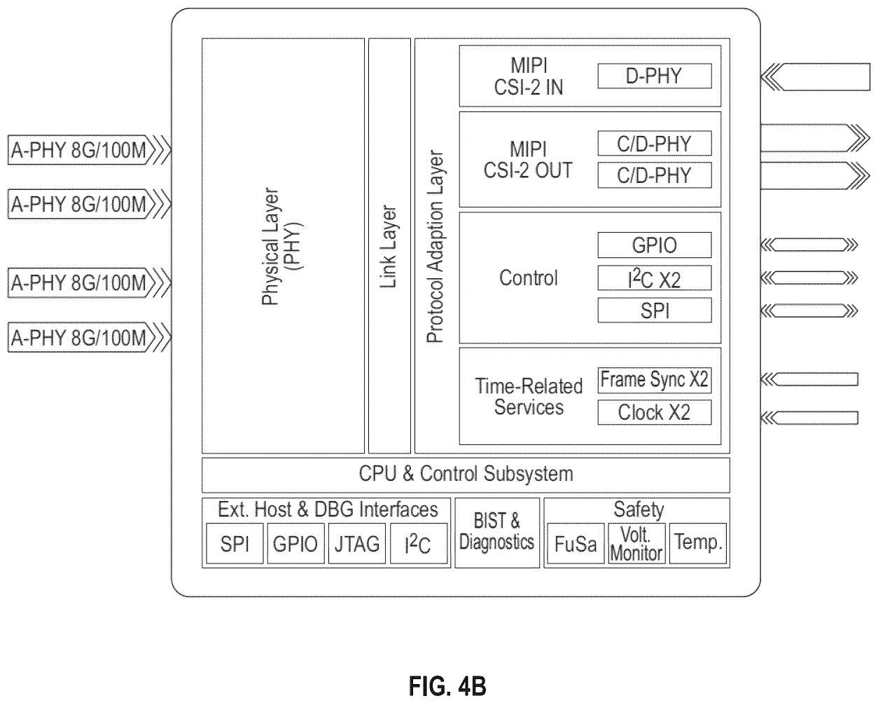

FIG. 4A illustrates an exemplary encoder, in accordance with some examples. The encoder may be used as the encoder 214 of the system 200 in FIG. 2. In some examples, the encoder is implemented as an A-PHY serializer. FIG. 4B illustrates an exemplary decoder, in accordance with some examples. The decoder may be used as the decoder 206 of the system 200 in FIG. 2. In some examples, the decoder is implemented as an A-PHY deserializer.

FIGS. 5A-G illustrate exemplary galvanic isolation schemes, in accordance with some examples. In some examples, the galvanic isolation component can ensure PAM-encoded data to be galvanically isolated. Galvanic isolation can be achieved more easily with PAM-encoded signals, because the signal frequency is lower. As shown in FIGS. 5A-G, the topology, location, and mechanism of the galvanic isolation component may vary, as long as it is capable of properly transmitting the PAM-encoded signals at the required frequency and providing the needed isolation voltage. There are multiple locations in the data transmission and processing path that the isolation can take place. In some examples, the isolation may occur in the cable and/or the connector. In some examples, an amplifier or equalizer is added before or after the transformer. In some examples, the isolation is achieved using a conversion to an optical transceiver and transmission through an optical fiber. In some examples, the isolation is achieved using a conversion to a GHz signal (millimeter-wave four-level pulse amplitude modulation (PAM4) signal). In some examples, the surgical imaging device contains an amplifier to increase the signal quality.

In FIG. 5A, the galvanic isolation component 502 is located between an encoder 504 (e.g., encoder 214 in FIG. 2) for generating PAM-encoded surgical imaging data and a decoder 506 (e.g., decoder 206 in FIG. 2) for decoding the PAM-encoded surgical imaging data. The cable 508 (e.g., twisted pair cable) for transmitting the PAM-encoded surgical imaging data can connect to the galvanic isolation component 502 directly. The galvanic isolation component 502 can comprise one or more Ethernet transformers, which can be designed for PAM-encoded signals. In some examples, the length of the cable may be short enough relative to the specified maximum supported cable length (e.g., 15 meters for A-PHY) such that there may be enough signal strength for the PAM-encoded surgical imaging data to go through the galvanic isolation component 502 without any retiming or redriving.

FIG. 5B illustrates a similar galvanic isolation scheme as FIG. 5A with magnetic isolation between the encoder 504 and the decoder 506, in accordance with some examples. The cable 508 connects a surgical imaging device (which comprises image sensors 503 and the encoder 504) and a CCU, which comprises the decoder 506. An equalizer or retimer 510 can be provided in the CCU to help boost the signal to compensate for the losses caused by the galvanic isolation component 502. In some examples, the equalizer or retimer 510 may be designed for a specific type of PAM-encoded signal (e.g., PAM4, PAM8, etc.).

FIG. 5C illustrates a similar isolation scheme as FIG. 5B, but with the equalizer or retimer 510 located in the surgical imaging device (e.g., a camera head).

FIG. 5D illustrates an exemplary galvanic isolation scheme in which the PAM-encoded surgical imaging data first passes through the decoder 506 before being isolated by the galvanic isolation component 502, in accordance with some examples. If A-PHY is used for the PAM-encoded surgical imaging data, the output of the decoder 506 is a MIPI CSI-2 signal. The MIPI CSI-2 signal may be converted to LVDS and then isolated using an LVDS isolator chip. In some examples, the PAM-encoded signal can be decoded directly into LVDS, or any NRZ signal.

The galvanic isolation in FIGS. 5A-C may be achieved with an isolation transformer or with a millimeter wave CMOS transceiver/contactless connector. In FIG. 5D, the isolation occurs after the PAM-encoded signal has been decoded into a MIPI CSI-2 signal. MIPI signals may be more difficult to isolate because the protocol has high-and low-frequency components. In some examples, the MIPI signal is translated into LVDS and then to an LVDS isolator chip. LVDS isolator chips typically have transformers for each channel. In some examples, the PAM-encoded signal can be decoded directly into LVDS, or any NRZ signal.

In FIGS. 5A-D, the DC-to-DC power isolation shares the same boundary and location as the video path isolation.

FIG. 5E illustrates how DC power can be sent from a CCU to the surgical imaging device (which comprises image sensors 503 and the encoder 504), in accordance with some examples. In some examples, the DC power can be sent from the CCU to the surgical imaging device by being injected into the center tap of the transformer. At the surgical imaging device, filters can be used to separate the high and low frequency components. The DC offset that was applied to the differential pair at the transformer can be used as power in the surgical imaging device. The high frequency PAM-encoded signal can be AC coupled into the differential pair.

FIGS. 5F and 5G provide more detailed views of power injection with a coaxial cable with isolation. The plan shown in FIG. 5D may also operate with power injection because the DC voltage may be coupled directly into the cable 508 between the encoders 504 with inductors. DC power in the CCU can be isolated using DC/DC isolation converters.

The isolation and power injection scheme in FIG. 5B can operate with both coaxial and twisted pair cables. The DC power injection can occur on the cable side of the equalizer/re-timer 510. The isolation and power injection scheme in FIG. 5D can also operate with both coaxial and twisted pair cables. The isolation and power injection scheme in FIG. 5F can operate with coaxial cables. The power injection occurs at the transformer. FIG. 5G also illustrates a coaxial implementation with power transmission. In this scheme, the video is converted from single-ended to differential before going to the transformer. The DC power injection occurs on the camera single-ended side of the conversion.

### Example System for Use in Generating Imaging Data

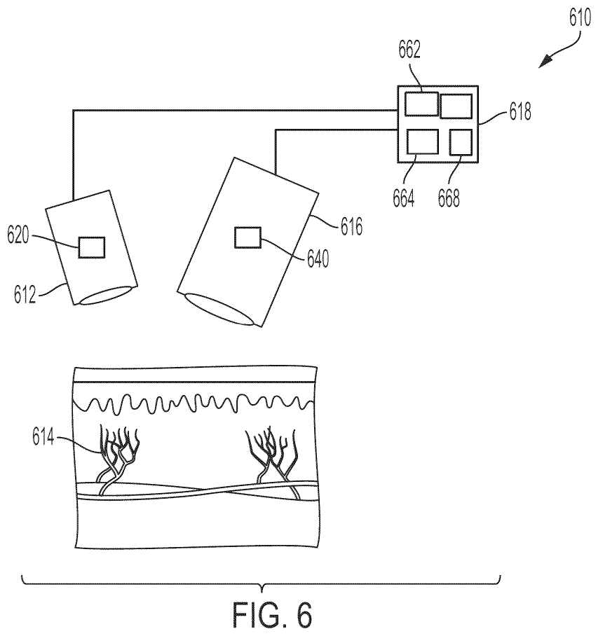

A system for collecting medical imaging data, such as system 10 of FIG. 1A, may include one or more imaging systems for acquiring a time series of images of tissue (e.g., a time series of fluorescence images, a time series of white light images, etc.). In some examples, an imaging system is a fluorescence imaging system. FIG. 6 is a schematic example of a fluorescence imaging system 610, according to some examples. The fluorescence imaging system 610 comprises a light source 612 to illuminate the tissue of the subject to induce fluorescence emission from a fluorescence imaging agent 614 in the tissue of the subject (e.g., in blood, in urine, in lymph fluid, in spinal fluid or other body fluids or tissues), an image acquisition assembly 616 arranged for generating the time series and/or the subject time series of fluorescence images from the fluorescence emission, and a processor assembly 618 arranged for processing the generated time series/subject time series of fluorescence images. The processor assembly 618 may include memory 668 with instructions thereon, a processor module 662 arranged for executing the instructions on memory 668 to process the time series and/or subject time series of fluorescence images, and a data storage module 664 to store the unprocessed and/or processed time series and/or subject time series of fluorescence images. In some variations, the memory 668 and data storage module 664 may be embodied in the same storage medium, while in other variations the memory 668 and the data storage module 664 may be embodied in different storage mediums. The system 610 may further include a communication module 666 for transmitting images and other data, such as some or all of the time series/subject time series of fluorescence images or other input data, spatial maps, subject spatial maps, and/or a tissue numerical value (quantifier), to an imaging data processing hub.

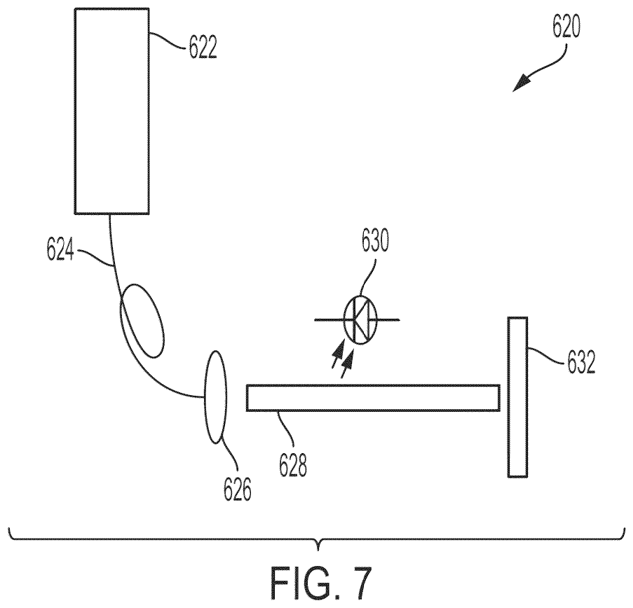

In this example, the light source 612 includes an illumination module 620. Illumination module 620 may include a fluorescence excitation source arranged for generating an excitation light having a suitable intensity and a suitable wavelength for exciting the fluorescence imaging agent 614. As shown in FIG. 7, the illumination module 620 may comprise a laser diode 622 (e.g., which may comprise, for example, one or more fiber-coupled diode lasers) arranged for providing an excitation light to excite the fluorescence imaging agent (not shown) in tissue of the subject. Examples of other sources of the excitation light which may be used in various examples include one or more LEDs, arc lamps, or other illuminant technologies of sufficient intensity and appropriate wavelength to excite the fluorescence imaging agent in the tissue. For example, excitation of the fluorescence imaging agent in blood, wherein the fluorescence imaging agent is a fluorescence dye with near infra-red excitation and emission characteristics, may be performed using one or more 793 nm, conduction-cooled, single bar, fiber-coupled laser diode modules from DILAS Diode Laser Co, Germany.

The light output from the light source 612 may be projected through one or more optical elements to shape and guide the output being used to illuminate the tissue area of interest. The optical elements may include one or more lenses, light guides, and/or diffractive elements so as to ensure a flat field over substantially the entire field of view of the image acquisition assembly 616. The fluorescence excitation source may be selected to emit at a wavelength close to the absorption maximum of the fluorescence imaging agent 614 (e.g., indocyanine green (ICG), etc.). For example, as shown in FIG. 7, the output 624 from the laser diode 622 may be passed through one or more focusing lenses 626, and then through a homogenizing light pipe 628 such as, for example, light pipes commonly available from Newport Corporation, USA. Finally, the light may be passed through an optical diffractive element 632 (e.g., one or more optical diffusers) such as, for example, ground glass diffractive elements also available from Newport Corporation, USA. Power to the laser diode 622 may be provided by, for example, a high-current laser driver such as those available from Lumina Power Inc. USA. The laser may optionally be operated in a pulsed mode during the image acquisition process. An optical sensor such as a solid state photodiode 630 may be incorporated into the illumination module 620 and may sample the illumination intensity produced by the illumination module 620 via scattered or diffuse reflections from the various optical elements. In some variations, additional illumination sources may be used to provide guidance when aligning and positioning the module over the area of interest.

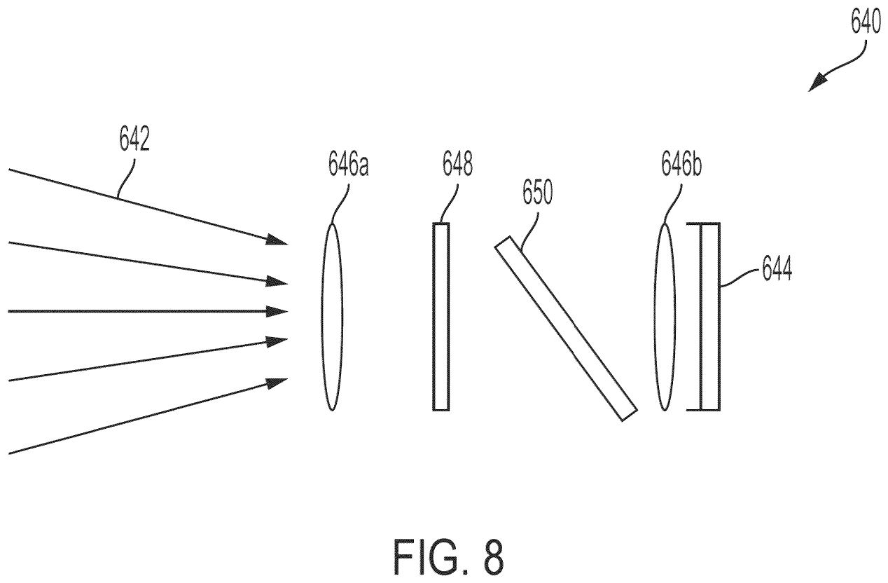

Referring again to FIG. 6, in this example, the image acquisition assembly 616 can be a component of a fluorescence imaging system 610 configured to acquire the time series and/or subject time series of fluorescence images from the fluorescence emission from the fluorescence imaging agent 614. The image acquisition assembly 616 may include a camera module 640, which may include an imaging device, such as endoscopic camera 13 of FIG. 1A, open-field imaging device 60 of FIG. 1B, and imaging device 201 of FIGS. 2A and 2B, connected to an imaging controller, such as imaging controller 203, via a cable, here an authenticable cable with memory, such as cable 202. As shown in FIG. 8, the camera module 640 may acquire images of the fluorescence emission 642 from the fluorescence imaging agent in the tissue by using a system of imaging optics (e.g., 646a, 646b, 648 and 650) to collect and focus the fluorescence emission onto an image sensor assembly 644. The image sensor assembly 644 may comprise at least one 2D solid state image sensor. The solid state image sensor may be a charge coupled device (CCD), a CMOS sensor, a CID or similar 2D sensor technology. The charge that results from the optical signal transduced by the image sensor assembly 644 is converted to an electrical video signal, which includes both digital and analog video signals, by the appropriate read-out and amplification electronics in the camera module 640.

According to an exemplary variation of a fluorescent imaging system, the light source may provide an excitation wavelength of about 800 nm +/−10 nm, and the image acquisition assembly uses emission wavelengths of >820 nm with NIR-compatible optics for, for example, ICG fluorescence imaging. In an exemplary example, the NIR-compatible optics may include a CCD monochrome image sensor having a GigE standard interface and a lens that is compatible with the sensor with respect to optical format and mount format (e.g., C/CS mount).

The processor module 662 may comprise any computer or computing means such as, for example, a tablet, laptop, desktop, networked computer, or dedicated standalone microprocessor. For instance, the processor module 662 may include one or more central processing units (CPU). In an exemplary example, the processor module 662 is a quad-core, 2.5 GHz processor with four CPUs where each CPU is a microprocessor such as a 64-bit microprocessor (e.g., marketed as INTEL Core i3, i5, or i7, or in the AMD Core FX series). However, in other examples, the processor module 662 may be any suitable processor with any suitable number of CPUs and/or other suitable clock speed. In some examples, the processor module 662 may include one or more Field Programmable Gate Arrays (FPGAs), one or more graphics processing units (GPUs), one or more application-specific integrated circuits (ASICs), one or more digital signal processors (DSPs), or any combination thereof.

Inputs for the processor module 662 may be taken, for example, from the image sensor 644 of the camera module 640 shown in FIG. 8, from the solid state photodiode 630 in the illumination module 620 in FIG. 7, and/or from any external control hardware such as a footswitch or remote-control. Output is provided to the laser diode driver and optical alignment aids. As shown in the example of FIG. 6, the processor assembly 618 may have a data storage module 664 with the capability to save the time series/subject time series of images, or data representative thereof, or other input data to a tangible non-transitory computer readable medium such as, for example, internal memory (e.g. a hard disk or flash memory), so as to enable recording and processing of acquired data. In some variations, the processor module 662 may have an internal clock to enable control of the various elements and ensure correct timing of illumination and sensor shutters. In some variations, the processor module 662 may also provide user input and graphical display of outputs. The fluorescence imaging system may optionally be configured with a communication unit 666, such as a wired or wireless network connection or video output connection for transmitting the time series of fluorescence images as they are being acquired or played back after recording. The communication unit 666 may additionally or alternatively transmit processed data, such as a spatial map, a subject spatial map, and/or tissue numerical value.

In operation of the exemplary system described in FIGS. 6-8, the subject is positioned relative to fluorescence imaging system 610 such that an area of interest (e.g., target tissue region) is located beneath the light source 612 and the image acquisition assembly 616 such that the illumination module 620 of light source 612 produces a substantially uniform field of illumination across substantially the entire area of interest. In some variations, prior to the administration of the fluorescence imaging agent 614 to the subject, an image may be acquired of the area of interest for the purposes of background deduction. To acquire fluorescence images/subject fluorescence images, the operator of the fluorescence imaging system 610 may initiate the acquisition of the time series/subject time series of fluorescence images by depressing a remote switch or foot-control, or via a keyboard (not shown) connected to the processor assembly 618. As a result, the light source 612 is turned on and the processor assembly 618 begins recording the fluorescence image data/subject fluorescence image data provided by the image acquisition assembly 616. When operating in the pulsed mode of the example, the image sensor 644 in the camera module 640 is synchronized to collect fluorescence emission following the laser pulse produced by the diode laser 622 in the illumination module 620. In this way, maximum fluorescence emission intensity is recorded, and signal-to-noise ratio is optimized. In this example, the fluorescence imaging agent 614 is administered to the subject and delivered to the area of interest via arterial flow. Acquisition of the time series/subject time series of fluorescence images is initiated, for example, shortly after administration of the fluorescence imaging agent 614, and the time series of fluorescence images from substantially the entire area of interest is acquired throughout the ingress of the fluorescence imaging agent 614. The fluorescence emission from the region of interest is collected by the collection optics of the camera module 640. Residual ambient and reflected excitation light is attenuated by subsequent optical elements (e.g., optical element 650 in FIG. 8 which may be a filter) in the camera module 640 so that the fluorescence emission can be acquired by the image sensor assembly 644 with minimal interference by light from other sources.

In some variations, following the acquisition or generation of the time series/subject time series of fluorescence images, the processor assembly 618 (e.g., processor module 662 or other processor) may then be initiated to execute instructions stored on memory 668 and process the imaging data before transmission to the imaging data processing system. The system 610 may transmit, via connection 666, the spatial map/subject spatial map and/or any clinical correlations or diagnosis derived therefrom or both for display to the user in a composite display feed as, for example, a grayscale or false color image, and/or stored for subsequent use.

A computer program product, such as a tangible non-transitory computer readable medium having computer-executable (readable) program code embedded thereon, may provide instructions for causing one or more processors to, when executing the instructions, perform one or more of the methods described herein. Program code can be written in any appropriate programming language and delivered to the processor in many forms, including, for example, but not limited to information permanently stored on non-writeable storage media (e.g., read-only memory devices such as ROMs, CD-ROM disks, etc.), information alterably stored on writeable storage media (e.g., hard drives or the like), information conveyed to the processor through communication media, such as a local area network, a public network such as the Internet, or any type of media suitable for storing electronic instruction. When carrying computer readable instructions that implement the various examples of the methods described herein, such computer readable media represent examples of various examples. In various examples, the tangible non-transitory computer readable medium comprises all computer-readable media, and the present invention scope is limited to computer readable media wherein the media is both tangible and non-transitory.

A kit may include any part of the systems described herein and the fluorescence imaging agent such as, for example, a fluorescence dye such as ICG or any suitable fluorescence imaging agent. In further aspects, a kit may include a tangible non-transitory computer readable medium having computer-executable (readable) program code embedded thereon that may provide instructions for causing one or more processors, when executing the instructions, to perform one or more of the methods for characterizing tissue and/or predicting clinical data described herein. The kit may include instructions for use of at least some of its components (e.g., for using the fluorescence imaging agent, for installing the computer-executable (readable) program code with instructions embedded thereon, etc.). In yet further aspects, there is provided a fluorescence imaging agent such as, for example, a fluorescence dye for use in in the methods and systems described herein. In further variations, a kit may include any part of or the entire system described herein and a fluorescence agent such as, for example, a fluorescence dye such as ICG, or any other suitable fluorescence agent, or a combination of fluorescence agents.

### Example Imaging Agents for Use in Generating Imaging Data

According to some examples, in fluorescence medical imaging applications, the imaging agent is a fluorescence imaging agent such as, for example, ICG dye. The fluorescence imaging agent, such as ICG, may be pre-administered to the subject, prior to performing the measurement of signal intensity arising from the fluorescence imaging agent. ICG, when administered to the subject, binds with blood proteins and circulates with the blood in the tissue. The fluorescence imaging agent (e.g., ICG) may be administered to the subject as a bolus injection (e.g., into a vein or an artery) in a concentration suitable for imaging such that the bolus circulates in the vasculature and traverses the microvasculature. In other examples in which multiple fluorescence imaging agents are used, such agents may be administered simultaneously, e.g. in a single bolus, or sequentially in separate boluses. The fluorescence imaging agents may be pre-administered to the subject, prior to performing the measurement of signal intensity arising from the fluorescence imaging agent. In some examples, the fluorescence imaging agent may be administered by a catheter. In certain examples, the fluorescence imaging agent may be administered less than an hour in advance of performing the measurement of signal intensity arising from the fluorescence imaging agent. For example, the fluorescence imaging agent may be administered to the subject less than 30 minutes in advance of the measurement. In yet other examples, the fluorescence imaging agent may be administered at least 30 seconds in advance of performing the measurement. In still other examples, the fluorescence imaging agent may be administered contemporaneously with performing the measurement.

According to some examples, the fluorescence imaging agent may be administered in various concentrations to achieve a desired circulating concentration in the blood. For example, in examples where the fluorescence imaging agent is ICG, it may be administered at a concentration of about 2.5 mg/mL to achieve a circulating concentration of about 5 μM to about 10 μM in blood. In various examples, the upper concentration limit for the administration of the fluorescence imaging agent is the concentration at which the fluorescence imaging agent becomes clinically toxic in circulating blood, and the lower concentration limit is the instrumental limit for acquiring the signal intensity data arising from the fluorescence imaging agent circulating with blood to detect the fluorescence imaging agent. In various other examples, the upper concentration limit for the administration of the fluorescence imaging agent is the concentration at which the fluorescence imaging agent becomes self-quenching. For example, the circulating concentration of ICG may range from about 2 μM to about 10 mM. Thus, in one aspect, the method comprises the step of administration of the imaging agent (e.g., a fluorescence imaging agent) to the subject and acquisition of the signal intensity data (e.g., video) prior to processing the signal intensity data according to the various examples. In another aspect, the method excludes any step of administering the imaging agent to the subject.

According to some examples, a suitable fluorescence imaging agent for use in fluorescence imaging applications to generate fluorescence image data is an imaging agent which can circulate with the blood (e.g., a fluorescence dye which can circulate with, for example, a component of the blood such as lipoproteins or serum plasma in the blood) and transit vasculature of the tissue (i.e., large vessels and microvasculature), and from which a signal intensity arises when the imaging agent is exposed to appropriate light energy (e.g., excitation light energy, or absorption light energy). In various examples, the fluorescence imaging agent comprises a fluorescence dye, an analogue thereof, a derivative thereof, or a combination of these. A fluorescence dye includes any non-toxic fluorescence dye. In certain examples, the fluorescence dye optimally emits fluorescence in the near-infrared spectrum. In certain examples, the fluorescence dye is or comprises a tricarbocyanine dye. In certain examples, the fluorescence dye is or comprises ICG, methylene blue, or a combination thereof. In other examples, the fluorescence dye is or comprises fluorescein isothiocyanate, rhodamine, phycoerythrin, phycocyanin, allophycocyanin, o-phthaldehyde, fluorescamine, rose Bengal, trypan blue, fluoro-gold, or a combination thereof, excitable using excitation light wavelengths appropriate to each dye. In some examples, an analogue or a derivative of the fluorescence dye may be used. For example, a fluorescence dye analog or a derivative includes a fluorescence dye that has been chemically modified, but still retains its ability to fluoresce when exposed to light energy of an appropriate wavelength.

In various examples, the fluorescence imaging agent may be provided as a lyophilized powder, solid, or liquid. In certain examples, the fluorescence imaging agent may be provided in a vial (e.g., a sterile vial), which may permit reconstitution to a suitable concentration by administering a sterile fluid with a sterile syringe. Reconstitution may be performed using any appropriate carrier or diluent. For example, the fluorescence imaging agent may be reconstituted with an aqueous diluent immediately before administration. In various examples, any diluent or carrier which will maintain the fluorescence imaging agent in solution may be used. As an example, ICG may be reconstituted with water. In some examples, once the fluorescence imaging agent is reconstituted, it may be mixed with additional diluents and carriers. In some examples, the fluorescence imaging agent may be conjugated to another molecule, such as a protein, a peptide, an amino acid, a synthetic polymer, or a sugar, for example to enhance solubility, stability, imaging properties, or a combination thereof. Additional buffering agents may optionally be added including Tris, HCl, NaOH, phosphate buffer, and/or HEPES.

A person of skill in the art will appreciate that, although a fluorescence imaging agent was described above in detail, other imaging agents may be used in connection with the systems, methods, and techniques described herein, depending on the optical imaging modality. Such fluorescence agents may be administered into body fluid (e.g., lymph fluid, spinal fluid) or body tissue.

In some variations, the fluorescence imaging agent used in combination with the methods, systems and kits described herein may be used for blood flow imaging, tissue perfusion imaging, lymphatic imaging, or a combination thereof, which may performed prior to, during or after an invasive surgical procedure, a minimally invasive surgical procedure, a non-invasive surgical procedure, or a combination thereof. The method of blood flow imaging, tissue perfusion imaging, lymphatic imaging, or a combination thereof, per se may exclude any invasive surgical step. Examples of invasive surgical procedure which may involve blood flow and tissue perfusion include a cardiac-related surgical procedure (e.g., CABG on pump or off pump) or a reconstructive surgical procedure. An example of a non-invasive or minimally invasive procedure includes wound (e.g., chronic wound such as for example pressure ulcers) treatment and/or management. In this regard, for example, a change in the wound over time, such as a change in wound dimensions (e.g., diameter, area), or a change in tissue perfusion in the wound and/or around the peri-wound, may be tracked over time with the application of the methods and systems. Examples of lymphatic imaging include identification of one or more lymph nodes, lymph node drainage, lymphatic mapping, or a combination thereof. In some variations such lymphatic imaging may relate to the female reproductive system (e.g., uterus, cervix, vulva).

In variations relating to cardiac applications, the imaging agent(s) (e.g., ICG alone or in combination with another imaging agent) may be injected intravenously through, for example, the central venous line, bypass pump and/or cardioplegia line to flow and/or perfuse the coronary vasculature, microvasculature and/or grafts. ICG may be administered as a dilute ICG/blood/saline solution down the grafted vessel such that the final concentration of ICG in the coronary artery is approximately the same or lower as would result from injection of about 2.5 mg (i.e., 1 ml of 2.5 mg/ml) into the central line or the bypass pump. The ICG may be prepared by dissolving, for example, 25 mg of the solid in 10 ml sterile aqueous solvent, which may be provided with the ICG by the manufacturer. One milliliter of the ICG solution may be mixed with 500 ml of sterile saline (e.g., by injecting 1 ml of ICG into a 500 ml bag of saline). Thirty milliliters of the dilute ICG/saline solution may be added to 10 ml of the subject's blood, which may be obtained in an aseptic manner from the central arterial line or the bypass pump. ICG in blood binds to plasma proteins and facilitates preventing leakage out of the blood vessels. Mixing of ICG with blood may be performed using standard sterile techniques within the sterile surgical field. Ten ml of the ICG/saline/blood mixture may be administered for each graft. Rather than administering ICG by injection through the wall of the graft using a needle, ICG may be administered by means of a syringe attached to the (open) proximal end of the graft. When the graft is harvested surgeons routinely attach an adaptor to the proximal end of the graft so that they can attach a saline filled syringe, seal off the distal end of the graft and inject saline down the graft, pressurizing the graft and thus assessing the integrity of the conduit (with respect to leaks, side branches etc.) prior to performing the first anastomosis. In other variations, the methods, dosages or a combination thereof as described herein in connection with cardiac imaging may be used in any vascular and/or tissue perfusion imaging applications.

Lymphatic mapping is an important part of effective surgical staging for cancers that spread through the lymphatic system (e.g., breast, gastric, gynecological cancers). Excision of multiple nodes from a particular node basin can lead to serious complications, including acute or chronic lymphedema, paresthesia, and/or seroma formation, when in fact, if the sentinel node is negative for metastasis, the surrounding nodes will most likely also be negative. Identification of the tumor draining lymph nodes (LN) has become an important step for staging cancers that spread through the lymphatic system in breast cancer surgery for example. LN mapping involves the use of dyes and/or radiotracers to identify the LNs either for biopsy or resection and subsequent pathological assessment for metastasis. The goal of lymphadenectomy at the time of surgical staging is to identify and remove the LNs that are at high risk for local spread of the cancer. Sentinel lymph node (SLN) mapping has emerged as an effective surgical strategy in the treatment of breast cancer. It is generally based on the concept that metastasis (spread of cancer to the axillary LNs), if present, should be located in the SLN, which is defined in the art as the first LN or group of nodes to which cancer cells are most likely to spread from a primary tumor. If the SLN is negative for metastasis, then the surrounding secondary and tertiary LN should also be negative. The primary benefit of SLN mapping is to reduce the number of subjects who receive traditional partial or complete lymphadenectomy and thus reduce the number of subjects who suffer from the associated morbidities such as lymphedema and lymphocysts.

The current standard of care for SLN mapping involves injection of a tracer that identifies the lymphatic drainage pathway from the primary tumor. The tracers used may be radioisotopes (e.g. Technetium-99 or Tc-99m) for intraoperative localization with a gamma probe. The radioactive tracer technique (known as scintigraphy) is limited to hospitals with access to radioisotopes require involvement of a nuclear physician and does not provide real-time visual guidance. A colored dye, isosulfan blue, has also been used, however this dye cannot be seen through skin and fatty tissue. In addition, blue staining results in tattooing of the breast lasting several months, skin necrosis can occur with subdermal injections, and allergic reactions with rare anaphylaxis have also been reported. Severe anaphylactic reactions have occurred after injection of isosulfan blue (approximately 2% of patients). Manifestations include respiratory distress, shock, angioedema, urticarial and pruritus. Reactions are more likely to occur in subjects with a history of bronchial asthma, or subjects with allergies or drug reactions to triphenylmethane dyes. Isosulfan blue is known to interfere with measurements of oxygen saturation by pulse oximetry and methemoglobin by gas analyzer. The use of isosulfan blue may result in transient or long-term (tattooing) blue coloration.

In contrast, fluorescence imaging in accordance with the various examples for use in SLN visualization, mapping, facilitates direct real-time visual identification of a LN and/or the afferent lymphatic channel intraoperatively, facilitates high-resolution optical guidance in real-time through skin and fatty tissue, visualization of blood flow, tissue perfusion or a combination thereof.

In some variations, visualization, classification or both of lymph nodes during fluorescence imaging may be based on imaging of one or more imaging agents, which may be further based on visualization and/or classification with a gamma probe (e.g., Technetium Tc-99m is a clear, colorless aqueous solution and is typically injected into the periarcolar area as per standard care), another conventionally used colored imaging agent (isosulfan blue), and/or other assessment such as, for example, histology. The breast of a subject may be injected, for example, twice with about 1% isosulfan blue (for comparison purposes) and twice with an ICG solution having a concentration of about 2.5 mg/ml. The injection of isosulfan blue may precede the injection of ICG or vice versa. For example, using a TB syringe and a 30 G needle, the subject under anesthesia may be injected with 0.4 ml (0.2 ml at each site) of isosulfan blue in the periarcolar area of the breast. For the right breast, the subject may be injected at 12 and 9 o'clock positions and for the left breast at 12 and 3 o'clock positions. The total dose of intradermal injection of isosulfan blue into each breast may be about 4.0 mg (0.4 ml of 1% solution: 10 mg/ml). In another exemplary variation, the subject may receive an ICG injection first followed by isosulfan blue (for comparison). One 25 mg vial of ICG may be reconstituted with 10 ml sterile water for injection to yield a 2.5 mg/ml solution immediately prior to ICG administration. Using a TB syringe and a 30G needle, for example, the subject may be injected with about 0.1 ml of ICG (0.05 ml at each site) in the periareolar area of the breast (for the right breast, the injection may be performed at 12 and 9 o'clock positions and for the left breast at 12 and 3 o'clock positions). The total dose of intradermal injection of ICG into each breast may be about 0.25 mg (0.1 ml of 2.5 mg/ml solution) per breast. ICG may be injected, for example, at a rate of 5 to 10 seconds per injection. When ICG is injected intradermally, the protein binding properties of ICG cause it to be rapidly taken up by the lymph and moved through the conducting vessels to the LN. In some variations, the ICG may be provided in the form of a sterile lyophilized powder containing 25 mg ICG with no more than 5% sodium iodide. The ICG may be packaged with aqueous solvent consisting of sterile water for injection, which is used to reconstitute the ICG. In some variations the ICG dose (mg) in breast cancer sentinel lymphatic mapping may range from about 0.5 mg to about 10 mg depending on the route of administration. In some variations, the ICG does may be about 0.6 mg to about 0.75 mg, about 0.75 mg to about 5 mg, about 5 mg to about 10 mg. The route of administration may be for example subdermal, intradermal (e.g., into the periarcolar region), subarcolar, skin overlaying the tumor, intradermal in the arcola closest to tumor, subdermal into arcola, intradermal above the tumor, periarcolar over the whole breast, or a combination thereof. The injections may be prior to visualization and/or classification. The NIR fluorescent positive LNs (e.g., using ICG) may be represented as a black and white NIR fluorescence image(s) for example and/or as a full or partial color (white light) image, full or partial desaturated white light image, an enhanced colored image, an overlay (e.g., fluorescence with any other image), a composite image (e.g., fluorescence incorporated into another image) which may have various colors, various levels of desaturation or various ranges of a color to highlight/visualize certain features of interest. Processing of the images may be further performed for further visualization and/or other analysis (e.g., quantification). The lymph nodes and lymphatic vessels may be visualized (e.g., intraoperatively, in real time) using fluorescence imaging systems and methods according to the various examples for ICG and SLNs alone or in combination with a gamma probe (Tc-99m) according to American Society of Breast Surgeons (ASBrS) practice guidelines for SLN biopsy in breast cancer patients. Fluorescence imaging for LNs may begin from the site of injection by tracing the lymphatic channels leading to the LNs in the axilla. Once the visual images of LNs are identified, LN mapping and identification of LNs may be done through incised skin, LN mapping may be performed until ICG visualized nodes are identified. The method of LN mapping per se may exclude any surgical step. For comparison, mapping with isosulfan blue may be performed until ‘blue’ nodes are identified. LNs identified with ICG alone or in combination with another imaging technique (e.g., isosulfan blue, and/or Tc-99m) may be labeled to be excised. Subject may have various stages of breast cancer (e.g., IA, IB, IIA).

In some variations, such as for example, in gynecological cancers (e.g., uterine, endometrial, vulvar and cervical malignancies), ICG may be administered interstitially for the visualization of lymph nodes, lymphatic channels, or a combination thereof. When injected interstitially, the protein binding properties of ICG cause it to be rapidly taken up by the lymph and moved through the conducting vessels to the SLN. ICG may be provided for injection in the form of a sterile lyophilized powder containing 25 mg ICG (e.g., 25 mg/vial) with no more than 5.0% sodium iodide. ICG may be then reconstituted with commercially available water (sterile) for injection prior to use. According to an example, a vial containing 25 mg ICG may be reconstituted in 20 ml of water for injection, resulting in a 1.25 mg/ml solution. A total of 4 ml of this 1.25 mg/ml solution is to be injected into a subject (4×1 ml injections) for a total dose of ICG of 5 mg per subject. The cervix may also be injected four (4) times with a 1 ml solution of 1% isosulfan blue 10 mg/ml (for comparison purposes) for a total dose of 40 mg. The injection may be performed while the subject is under anesthesia in the operating room. In some variations the ICG dose (mg) in gynecological cancer sentinel lymph node detection and/or mapping may range from about 0.1 mg to about 5 mg depending on the route of administration. In some variations, the ICG does may be about 0.1 mg to about 0.75 mg, about 0.75 mg to about 1.5 mg, about 1.5 mg to about 2.5 mg, about 2.5 mg to about 5 mg. The route of administration may be for example cervical injection, vulva peritumoral injection, hysteroscopic endometrial injection, or a combination thereof. In order to minimize the spillage of isosulfan blue or ICG interfering with the mapping procedure when LNs are to be excised, mapping may be performed on a hemi-pelvis, and mapping with both isosulfan blue and ICG may be performed prior to the excision of any LNs. LN mapping for Clinical Stage I endometrial cancer may be performed according to the NCCN Guidelines for Uterine Neoplasms, SLN Algorithm for Surgical Staging of Endometrial Cancer; and SLN mapping for Clinical Stage I cervical cancer may be performed according to the NCCN Guidelines for Cervical Neoplasms, Surgical/SLN Mapping Algorithm for Early-Stage Cervical Cancer. Identification of LNs may thus be based on ICG fluorescence imaging alone or in combination or co-administration with for a colorimetric dye (isosulfan blue) and/or radiotracer.

Visualization of lymph nodes may be qualitative and/or quantitative. Such visualization may comprise, for example, lymph node detection, detection rate, anatomic distribution of lymph nodes. Visualization of lymph nodes according to the various examples may be used alone or in combination with other variables (e.g., vital signs, height, weight, demographics, surgical predictive factors, relevant medical history and underlying conditions, histological visualization and/or assessment, Tc-99m visualization and/or assessment, concomitant medications). Follow-up visits may occur on the date of discharge, and subsequent dates (e.g., one month).

Lymph fluid comprises high levels of protein, thus ICG can bind to endogenous proteins when entering the lymphatic system. Fluorescence imaging (e.g., ICG imaging) for lymphatic mapping when used in accordance with the methods and systems described herein offers the following example advantages: high-signal to background ratio (or tumor to background ratio) as NIR does not generate significant autofluorescence, real-time visualization feature for lymphatic mapping, tissue definition (i.e., structural visualization), rapid excretion and elimination after entering the vascular system, and avoidance of non-ionizing radiation. Furthermore, NIR imaging has superior tissue penetration (approximately 5 to 10 millimeters of tissue) to that of visible light (1 to 3 mm of tissue). The use of ICG for example also facilitates visualization through the peritoneum overlying the para-aortic nodes. Although tissue fluorescence can be observed with NIR light for extended periods, it cannot be seen with visible light and consequently does not impact pathologic evaluation or processing of the LN. Also, florescence is easier to detect intra-operatively than blue staining (isosulfan blue) of lymph nodes. In other variations, the methods, dosages or a combination thereof as described herein in connection with lymphatic imaging may be used in any vascular and/or tissue perfusion imaging applications.

Tissue perfusion relates to the microcirculatory flow of blood per unit tissue volume in which oxygen and nutrients are provided to and waste is removed from the capillary bed of the tissue being perfused. Tissue perfusion is a phenomenon related to but also distinct from blood flow in vessels. Quantified blood flow through blood vessels may be expressed in terms that define flow (i.e., volume/time), or that define speed (i.e., distance/time). Tissue blood perfusion defines movement of blood through micro-vasculature, such as arterioles, capillaries, or venules, within a tissue volume. Quantified tissue blood perfusion may be expressed in terms of blood flow through tissue volume, namely, that of blood volume/time/tissue volume (or tissue mass). Perfusion is associated with nutritive blood vessels (e.g., micro-vessels known as capillaries) that comprise the vessels associated with exchange of metabolites between blood and tissue, rather than larger-diameter non-nutritive vessels. In some examples, quantification of a target tissue may include calculating or determining a parameter or an amount related to the target tissue, such as a rate, size volume, time, distance/time, and/or volume/time, and/or an amount of change as it relates to any one or more of the preceding parameters or amounts. However, compared to blood movement through the larger diameter blood vessels, blood movement through individual capillaries can be highly erratic, principally due to vasomotion, wherein spontaneous oscillation in blood vessel tone manifests as pulsation in erythrocyte movement. In some examples, blood flow and tissue perfusion imaging described herein in connection with the systems and methods may be used to image tumor tissue and differentiate such tissue from other tissue.

The foregoing description, for the purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated. For the purpose of clarity and a concise description, features are described herein as part of the same or separate examples, however, it will be appreciated that the scope of the invention may include examples having combinations of all or some of the features described.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A system for acquiring surgical imaging data, comprising:
a surgical imaging device for generating Pulse Amplitude Modulation (PAM)-encoded surgical imaging data, wherein:
the PAM-encoded surgical imaging data comprises a plurality of pulses, and
each pulse of the plurality of pulses is configured to have one of more than two amplitude levels;
an imaging processor for receiving data corresponding to the PAM-encoded surgical imaging data; and
a galvanic isolation component between the surgical imaging device and the imaging processor.

2. The system of claim 1, wherein each pulse of the plurality of pulses is configured to have one of four amplitude levels, one of eight amplitude levels, or one of sixteen amplitude levels.

3. The system of claim 1, wherein the PAM-encoded surgical imaging data is encoded and transmitted in accordance with the A-PHY standard.

4. The system claim 1, wherein the surgical imaging device comprises:
one or more imaging sensors for generating original surgical imaging data; and
an encoder for encoding the original surgical imaging data to obtain the PAM-encoded surgical imaging data.

5. The system of claim 4, wherein the encoder is a programmable device or an application-specific integrated circuit (ASIC).

6. The system of claim 1, wherein the surgical imaging device comprises one or more imaging sensors for generating the PAM-encoded surgical imaging data.

7. The system of claim 1, further comprising a cable for transmitting the PAM-encoded surgical imaging data from the surgical imaging device, wherein the cable comprises a transmission line.

8. The system of claim 7, wherein the transmission line is a twisted pair transmission line.

9. The system of claim 7, wherein the transmission line is a coaxial transmission line.

10. The system of claim 7, wherein the transmission line is configured to transmit power from the imaging processor to the surgical imaging device.

11. The system of claim 7, wherein the cable is configured to transmit a control signal from the imaging processor to the surgical imaging device.

12. The system of claim 11, wherein the control signal and the PAM-encoded signal are transmitted as a plurality of data packets along the transmission line.

13. The system of claim 12, wherein the plurality of packets have the same structure.

14. The system of claim 12, wherein at least one of the plurality of packets is configured to be retransmitted using a retransmission buffer.

15. The system of claim 7, wherein the system further comprises a light source, wherein the surgical imaging device is configured to output one or more light pulses, and wherein synchronization of the light source and the one or more light pulses is controlled by a synchronization signal on the transmission line.

16. The system of claim 1, wherein the galvanic isolation component comprises a magnetic transformer or one or more CMOS isolation circuits.

17. The system of claim 1, further comprising a decoder for decoding the PAM-encoded surgical imaging data.

18. The system of claim 17, wherein the galvanic isolation component is located between the surgical imaging device and the decoder.

19. The system of claim 17, wherein the galvanic isolation component is located between the decoder and the imaging processor.

20. The system of claim 1, wherein the surgical imaging data comprises at least one of pixel data and voxel data.
